(19) United States
(12) Patent Application Publication
(10) Pub. No.: US 2005/0065182 A1
(43) Pub. Date: Mar. 24, 2005

(54) Method and compositions for chronic wound care

(76) Inventor: Elisabeth S. Papazoglou, West Lafayette, IN (US)

Correspondence Address: Laurence T. Pearson, 2009 Dogwood Lane, Wilmington, DE, 19810, US

(21) Appl. No.: 10/667,835

(22) Filed: Sep. 23, 2003

(51) Int. Cl. A61K 31/445

(52) U.S. Cl. 514/316; 514/327

(57) ABSTRACT

A method for the treatment of chronic wounds in vertebrates, and compositions for use in the method, said compositions comprising synthetic antioxidants based on hindered piperidine or hindered phenol structures. The compositions optionally comprise carrier compounds that can include hydrogels.

### BACKGROUND OF THE INVENTION

1. Field of the Invention

The present invention relates to a method for promoting and accelerating healing in a chronic wound in vertebrates, including human beings, by topical administration of an antioxidant to the wound.

2. Description of Related Art

Wound Healing

Wound healing takes place via a series of biochemical and cellular events which result in the contraction, closing and healing of a wound. Normal wound healing is a balance of new tissue formation and destructive processes necessary to remove damaged tissue. For example, matrix metalloproteases (MMP's) have a role in the removal of damaged tissue, but can interfere with the growth of a new tissue environment.

Within this complex environment many points of regulation control the biological processes necessary to achieve normal wound repair. An alteration in any of these processes can lead to the formation of a chronic wound. In general, chronic wounds are characterized by a prolonged inflammatory phase, which ultimately results in elevated activity of MMP's (see for example Wall, S. J. et. al. Journal of Investigative Dermatology, 119, pp 91-98 (2002)) and the subsequent degradation of growth factors and other positive wound healing factors; the overall effect is impaired healing.

Protease excess has been detected extracellularly (A. B. Wysocki et. al., Journal of Investigative Dermatology, 101: pp64068 (1993)) and proteases have been implicated in all stages of wound repair and have been shown to play an important role in regulating the balance between tissue synthesis and tissue destruction (G. S. Schultz and B. A. Mast, Wounds, 10: pp 1F-9F (1998)). Thus, while controlled degradation is necessary for normal wound healing to proceed, excess or prolonged proteolytic activity is considered detrimental and thought to contribute to the chronicity of the wound. This hypothesis is supported by many investigators who have examined the protease profile of human chronic wound fluid (R. S. Kirsner et. al., Wounds 3: pp122-128 (1993) and M. Palolahti et. al., Experimental Dermatology, 2: pp29-37 (1993)). These studies have shown that elevated levels of proteases, particularly the matrix metalloproteases (MMPs), are present in all types of chronic wounds independent of the etiology.

Chronic wounds are also characterized by different levels of certain species relative to non chronic wounds. For example elevated levels of reactive oxygen species (ROS) such as hydrogen peroxide, the superoxide radical, and singlet oxygen, all of which have also been shown to directly cause elevated levels of MMP's, are seen. Depressed levels of nitrogen oxide are also seen in chronic wounds relative to acute wounds.

Even though ROS are constantly being produced in living organisms, wound healing is also known to be an event characterized by oxidative stress, in which elevated levels of ROS are seen (for example; D. Darr and 1. Fridovich, Journal of Investigative Dermatology, 102: pp 671-675 (1994)). Both hydrogen peroxide and superoxide have been implicated in elevation of the level of MMP in wounds (see J. Wenk et. al., Journal of Biological Chemistry, 274: pp 25869-25876, (1999)). Natural antioxidants (i.e. those synthesized in vivo) and Nitrogen oxide (NO) have been shown to be effective when applied to diabetic ulcers (for example, J. V. Boykin, Advances in Skin Wound Care, 13: pp169-174 (2000) and K. S. Bohl Masters et. al. Wound Repair and Regeneration, 10: pp286-294 (2002), and M. B. Witte et. al., Surgical Forum, 48: pp665-667 (1997)).

Growth factors, such as platelet-derived growth factor (PDGF), transforming growth factor-beta (TGF-β), vascular endothelial growth factor (VEGF), and epidermal growth factor (EGF), are pivotal in normal wound repair, driving cell migration, proliferation, protein synthesis, and extracellular matrix formation (for example “Molecular Biology of the Cell” 4^{th} edition, 2001, published by Garland Science, B. Alberts et al., p. 1019). Thus, the degradation of either the growth factors or the de novo granulation tissue by excess proteases in the chronic wound would result in delayed healing. This also may explain why growth factors can accelerate healing in acute wounds but have had limited success in the treatment of chronic wounds, even when used at high doses (C. O. Brantigan, Wounds, 8:78-90 (1996).

Chronic wounds, therefore, can be considered to exhibit an imbalance between tissue deposition stimulated by growth factors and tissue destruction mediated by proteases in which the balance favors the destructive processes. It is postulated that high protease activity present in chronic wounds degrades peptide growth factors, rendering them inactive (V. Falanga, Journal of Dermatology, 19:667-672 (1002)).

Thus, it is proposed that an alternate strategy to the addition of exogenous growth factors for the treatment of chronic wounds would be to modify the chronic wound environment by reducing the levels of proteases, thereby protecting endogenous growth factors and allowing normal wound repair to proceed.

Wound Management

Wound management must protect the wound from additional trauma and/or environmental factors that would delay the healing process. It may also require intervention in the healing process using externally applied agents if the healing process becomes abnormal in some way. Wound management usually consists of a combined systemic and local approach, including the use of antibiotics and the application of a suitable dressing.

Supplements to wound management protocols could include additives that modulate, and preferably reduce, the protease activity in the wound microenvironment, or mitigate the effect of MMP's. For example, platelet derived growth factor (PDGF) has been proven to be effective for the treatment of diabetic ulcers (see for example, M. A. M. Loots, et. al., European Journal of Cell Biology, 81, pp153-160 (2002))

In the present invention, we take advantage of ther fact that there are many low cost antioxidants available that are able to apply the NO radical in the form of a stable chemical form (the piperidinyl radical) to a substrate. Other chemical functionalities are also available to carry out the same function, which is to supply stable free radicals that have a controlling effect on oxidative species such as peroxides, superoxides and singlet oxygen.

Examples in the patent art of supplements to wound management protocols include treatment with polysacharrides, for example U.S. Pat. No. 5,468,737, in which the polysacharride is from aloe, U.S. Pat. No. 5,502,042, and U.S. Pat. No. 5,902,078 in which heparin—a polysacharride, is the supplement. In a further application of an aloe derivative, as a protectant for growth factor, U.S. Pat. No. 6,313,103 uses aloe pectin. U.S. Pat. No. 6,165,994 utilizes α-D glucan as a supplement.

U.S. Pat. No. 6,395,709 and U.S. Pat. No. 6,399,580 relate to cosmetic and therapeutic methods for stimulating tissue growth and/or enhancing moisturization and lubrication in mammalian epithelium, such as skin and mucous membrane. The methods comprises providing to an individual a composition comprising at least one inhibitor of β-glucosidase activity, a glycosphingolipid or the combination of a β-glucosidase inhibitor with a glycosphingolipid.

U.S. Pat. No. 6,573,249 describes the use of cromolyn with and without hyaluronic acid as a supplement.

The present invention describes the use of synthetic antioxidants to stabilize the wound environment (growth factors in the wound micro environment) by treatment of the wound with antioxidants, preferably in mixtures with surfactant in hydrogels. The use of synthetic antioxidants to stabilize sythetic polymers is well know in the art. For example The stabilization of synthetic polymers with hindered amines has been described for example in U.S. Pat. Nos. 4,086,204, 4,331,586, 4,335,242, 4,234,707, 4,459,395, 4,492,791, 5,204,473, EP-A-53 775, EP-A-357 223, EP-A-377 324, EP-A-462 069, EP-A-782 994 and GB-A-2 301 106.

Antioxidants of a particular structure have been disclosed in the art for treating various problems with skin and with wounds.

For example, U.S. Pat. No. 5,980,920 as useful for protecting skin against UV radiation.

The use of antoxidants in wound care is also described in U.S. Pat. No. 5,612,321 (hereinafter '321) assigned to Hercules, in which a hindered phenol moiety is grafted onto a polysaccharide chain. The grafted antixodant moiety is proposed to protect the rest of the chain from oxidation and hence degradation in molecular weight.

The invention of '321 requires specific synthesis and development in commercial quantities of a new molecule before commercial feasibility can be established, with all the cost that is associated with that process. There is therefore a need for an inexpensive solution to the problem of finding functional antioxidants for the treatment of wounds.

U.S. Pat. Nos. 5,658,956 and 6,329,343 and publications WO 96/03149 and WO 96/06640 disclose wound healing compositions that comprise antioxidants with pyrivic acid or derivatives and a mixture of fatty acids.

A large number of molecules exist that function as antioxidants for organic materials and that can be obtained very cheaply.

It is the intention of the present invention to disclose and claim the use of commonly and commercially available antioxidants that can be included in formulations for care of wounds. Without wishing to be bound by mechanism, it is possible that the mechanism of action of such antioxidants would be proection of polysaccarides, such as hyaluronic acid, that are helpful in the wound healing process, or modulation of the overall activity of MMP's in the wound microenvironment via supression of ROS.

### BRIEF SUMMARY OF THE INVENTION

The present invention is directed towards a method for the treatment, control, and healing of wounds, and in particular chronic wounds, and in particular chronic wounds that result from diabetes, by the application of compositions that comprise synthetic antioxidants. The compositions are preferably applied to the wound during the initial phase of healing, which is the first three days, when the wound microenvironment is developing.

The invention is also directed towards the compositions that are applied to wounds, and in particular chronic wounds, and in particular chronic wounds that result from diabetes.

### DETAILED DESCRIPTION OF THE INVENTION

### Classes of Antioxidants of the Present Invention

The general structure and mechanism of action of antioxidants is described in Chapters 1 and 2 of “Plastics Additives”, 5th edition, Hanser Gardner, 2001, ed. H. Zweifel, and incorporated herein by reference. In summary, antioxidants are generally characterized by the ability to form stable free radicals under appropriate environmental conditions. These stable free radicals then have the ability, within the matrix that they are stabilizing, to trap and remove other free radicals that may be participating in chemical reactions that are harmful to the substrate. In particular oxygen centered harmful free radicals are the target of commercial synthetic antioxidants.

### Definitions

“Active center” refers to the chemical group that produced the antioxidant action. Examples of active centers are the phenolic moiety in hindered phenols, such as 2,6-di tertbutylphenol; and the piperidine ring in hindered amines such as 2,2,6,6,-tetramethylpiperidine. Those skilled in the art will recognize that there are other types of active center not mentioned here that have antioxidant action.

“Pendant group” refers to the chemical moiety that is appended to the active center.

“Hindered amine” refers to a derivative of a 2,2,6,6-tetraalkylpiperidine and preferably 2,2,6,6-tetramethylpiperidine

“Hindered phenol” refers to a derivative of a 2,6-di-alkylphenol, and preferably 2,6-di-tert butylphenol.

“Surfactant” refers to a material which when used in an effective amount in a mixture allows the other components of the mixture to be dispersed in an aqueous base.

### EXAMPLES OF COMPOSITIONS OF THE PRESENT INVENTION

The wound care composition of the invention may contain various hindered phenol antioxidants, for example:

1. Alkylated monophenols, for example 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(.alpha.-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof.

2. Alkylthiomethylphenols, for example 2,4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonylphenol.

3. Hydroquinones and alkylated hydroquinones, for example 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis-(3,5-di-tert-butyl-4-hydroxyphenyl)adipate.

4. Tocopherols, for example α-tocopherol, β-tocopherol, γ-tocopherol, δ-tocopherol and mixtures thereof.

5. Hydroxylated thiodiphenyl ethers, for example 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis-(3,6-di-sec-amylphenol), 4,4′-bis-(2,6-dimethyl-4-hydroxyphenyl)disulfide.

6. Alkylidenebisphenols, for example 2,2′-methylenebis(6-tert-butyl-4-methylphenol), 2,2′-methylenebis(6-tert-butyl-4-ethylphenol), 2,2′-methylenebis[4-methyl-6-(.alpha.-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(.alpha.-methylbenzyl)-4-nonylphenol], 2,2′-methylenebis[6-(α, α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis-(3,5-di-tert-butyl-4-hydroxyphenyl)propane, 2,2-bis-(5-tert-butyl-4-hydroxy2-methylphenyl)-4-n-dodecylmercaptobutane, 1,1,5,5-tetra-(5-tert-butyl-4-hydroxy2-methylphenyl)pentane.

7. O-, N- and S-benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5di-tert-butyl-4-hydroxybenzylmercaptoacetate.

8. Hydroxybenzylated malonates, for example dioctadecyl-2,2-bis-(3,5-di-tert-butyl-2-hydroxybenzyl)-malonate, di-octadecyl-2-(3-tert-butyl-4-hydroxy-5-methylbenzyl)-malonate, di-dodecylmercaptoethyl-2,2-bis-(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

9. Aromatic hydroxybenzyl compounds, for example 1,3,5-tris-(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol.

10. Triazine Compounds, for example 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tis(3,5-di-tert-butyl-4-hydroxyphenoxy)-1,2,3-triazine, 1,3,5-tris-(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-tri azine, 1,3,5-tris(3,5-dicyclohexyl-4-hydroxybenzyl)isocyanurate.

11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl3,5-di-tert-butyl-4-hydroxybenzylphosphonate, dioctadecyl-5-tert-butyl-4-hydroxy3-methylbenzylphosphonate, the calcium salt of the monoethyl ester of 3,5-di-tert-butyl-4-hydroxybenzylphosphonic acid.

12. Acylaminophenols, for example 4-hydroxylauranilide, 4-hydroxystearanilide, octyl N-(3,5-di-tert-butyl-4-hydroxyphenyl)carbamate.

13. Esters of .beta.-(3,5di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

14. Esters of β-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

17. Amides of β-(3,5di-tert-butyl-4hydroxyphenyl)propionic acid e.g. N,N′-bis(3,5-di-tert-butyl-4-hydroxyphenylpropionyl)hexamethylenediamine, N,N′-bis(3,5-di-tert-butyl-4-hydroxyphenylpropionyl)trimethylenediamine, N,N′-bis(3,5-di-tert-butyl-4-hydroxyphenylpropionyl)hydrazine.

The invention may also comprise hindered amines, examples of which are:

bis(2,2,6,6-tetramethyl-4-piperidyl)sebacate,
bis(2,2,6,6-tetramethyl-4-piperidyl)succinate,
bis(1,2,2,6,6-pentamethyl-4-piperidyl)sebacate,
bis(1-octyloxy-2,2,6,6-tetramethyl-4-piperidyl)sebacate,
bis(1,2,2,6,6-pentamethyl-4-piperidyl) n-butyl-3,5-di-tert-butyl-4-hydroxybenzylmalonate,
the condensate of 1-(2-hydroxyethyl)-2,2,6,6-tetramethyl-4-hydroxypiperidine and succinic acid,
the condensate of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate,
tetrakis(2,2,6,6-tetramethyl-4-piperidyl)-1,2,3,4-butane-tetracarboxylate, 4-benzoyl-2,2,6,6-tetramethylpiperidine,
4-stearyloxy-2,2,6,6-tetramethylpiperidine, bis(1,2,2,6,6-pentamethylpiperidyl)-2-n-butyl-2-(2-hydroxy-3,5-di-tert-butyl-benzyl)malonate, 3-n-octyl-7,7,9,9-tetramethyl-1,3,8-triazaspiro[4.5]decan-2,4-dion, bis(1-octyloxy-2,2,6,6-tetramethylpiperidyl)sebacate,
bis(1-octyloxy-2,2,6,6-tetramethylpiperidyl)succinate,
the condensate of N,N′-bis-(2,2,6,6-tetramethyl-4-piperidyl)hexamethylenediamine and 4-morpholino-2,6-dichloro-1,3,5-triazine,
the condensate of 2-chloro-4,6-bis(4-n-butylamino-2,2,6,6-tetramethylpiperidyl)-1,3,5-triazine and 1,2-bis(3-aminopropylamino)ethane,
the condensate of 2-chloro-4,6-di-(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4. 5]decane-2,4-dione, 3-dodecyl-1-(2,2,6,6-tetramethyl-4-piperidyl)pyrrolidin-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine,
the condensation product of N,N′-bis(2,2,6,6-tetramethyl-4-piperidyl)hexamethylenediamine and 4-cyclohexylamino-2,6-dichloro-1,3,5-triazine,
the condensation product of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]);
N-(2,2,6,6-tetramethyl-4-piperidyl)-n-dodecylsuccinimid, N-(1,2,2,6,6-pentamethyl-4-piperidyl)-n-dodecylsuccinimid, 2-undecyl-7,7,9,9-tetramethyl-1-oxa-3,8-diaza-4-oxo-spiro[4,5]decane, and
the reaction product of 7,7,9,9-tetramethyl-2-cycloundecyl-1-oxa-3,8-diaza-4-oxospiro [4,5]decane andnd epichlorohydrin.

The invention also comprises other compounds that act as carriers. Examples of other compounds would be inert carriers. By “inert carriers” is meant compounds that function soley to carry the antioxidant to the wound environment and hold it there. Examples of inert carriers would be hydrogels comprising polyacryl amide, polymethacrylic acid, copolymers such as diacetone acrylamide (DAA) and hydroxyethyl acrylate (HEA), aliphatic polyesters based on lactic or glycolic acid, polyacrylonitril hydrogels, or polyvinyl alcohol hydrogels. Vitamin E and its derivatives, for example α-tocopherol, β-tocopherol, γ-tocopherol, δ-tocopherol and mixtures thereof, are also suitable for use as inert carriers.

### DESCRIPTION OF PREFERRED EMBODIMENTS

The preferred method of application of antioxidants to wounds is in the form of a hydrogel that comprises polyvinyl alcohol, or poly(2-hydroxyethylmethacrylate) or polyacrylamide, or polymethacrylic acid, or copolymers such as diacetone acrylamide (DM) and hydroxyethyl acrylate (HEA). Other suitable polymer matrices would be aliphatic polyesters based on lactic and glycolic acid, polyacrylonitrile hydrogels.

Blends of the antioxidants and carrier can be made by adding the required amount of ground antioxidant powder to water and dispersing the antioxidant powder by high speed mixing or ultrasonication. The dispersion is then gelled by mixing with polyvinyl alcohol pellets.

In an example of a preferred composition of the invention, a polyvinyl alcohol (PVA) hydrogel is prepared in the presence of tetrakis[methylene 3-(3′,5′-di-tert-butyl-4′-hydroxyphenyl)propionate]methane (sold by Great Lakes Chemical Company, Indianapolis, Ind. under the tradename Anox® 20). A suitable source of PVA pellets is Elvanol® 71-30 (Du Pont Company, Wilmington, Del.).

A hydrogel is prepared by the following steps:

I. A weight of Anox® 20 is ground in a laboratory or coffee grinder until a fine powder is obtained as judged visually.
II. The ground Anox 20 powder is dispersed using a high speed mixer into water at 24° C. (75° F.) at a concentration of 5% by weight.
III. Elvanol® 71-30 as received from the manufacturer at a concentration of 10% by weight water is sifted into the Anox® 20 suspension at 24° C. (75° F.).
IV. The slurry is stirred for 10 minutes.
V. The temperature is raised to 90° C. (194° F.) or above and stirring is continued at 90-95° C. (194-203° F.) until the resin is dissolved, generally 30-60 minutes.
VI. The gel is allowed to cool to room temperature.
   Solutions can be prepared in any vessel equipped with a high-speed agitator that produces a good vortex in the water. Vessels used for preparing or storing solutions of Elvanol® should be constructed of or lined with corrosion-resistant material such as stainless steel, Monel metal, tin, glass or wood. Heat should be supplied by steam jacket or coil, or by injecting live steam. Application of direct heat or flame should be avoided.

In further examples of the method of this invention, we note the fact that the art of solubilizing organic materials that are intrinsically insoluble in aqueous phases is described extensively in the patent literature. For example U.S. Pat. No. 5,234,695 assigned to Eastmen Kodak and hereby incorporated in its entirety by reference, describes the preparation of a soluble form of vitamin E, denoted vitamin E—TPGS. The product of the invention disclosed and claimed in U.S. Pat. No. 5,234,695 and described also in Eastman publication EFC-226A (1998) dissolves readily in water, and such a base of 10% or higher Vitamin E—TPGS dissolved in a dispersion of Anox® 20 is a suitable hydrogel for the method of the present invention.

Similarly, U.S. Pat. No. 5,358,560 and U.S. Pat. No. 500,256 assigned to Eastman Chemical and also incorprated herein in their entirety by reference describe methods and compositions for dispersing additives in water. An as an example of how such dispersions can be used in the present invention, in example 1 from the '560 patent a suspension of Anox® 20 is made from the following composition (numbers in %):
IRGANOX® 1010 antioxidant (85.9%) (from Ciba Geigy Corp, and equivalent to Anox® 20.)
EPOLENE E-14 wax (12.8%) (an emulsifiable polyethylene wax from Eastman Chemical Company having an acid number of 16).
ARLACEL 80 surfactant (0.835%) (a surfactant having an HLB of about 4 from ICI).
IGEPAL CO-surfactant (0.422%) (a surfactant having an HLB of about 13 from GAF).
Water is added to form suspension at approximately 35 wt % additive.

The powder raw materials are milled to an acceptable particle size distribution, if necessary. Each of the ingredients were weighed into a 4 ounce glass jar. Mixing the ingredients forms a water dispersible powder. Water was added and the bottle was shaken by hand or stirred with a magnetic stirrer.

For the purposes of the present invention, to this dispersion is added sufficient poly vinylalcohol pellets (Elvanol®71-30, Du Pont) with stirring and heating as described above, to make a gel of the required viscosity for application to a wound directly, or incorporation into a wound dressing.

The other examples of antioxidant dispersions given in U.S. Pat. No. 5,358,560 are suitable for use as precursors to the manufacture of the hydrogels of the present invention.

Similarly, U.S. Pat. No. 5,443,910 assigned to Eastman Chemical and incorporated herein in its entirety by reference also contains examples of the manufacture of aqueous emulsions of antioxidants that are suitable for use in the present invention in the same manner as described above, i.e. production of the emulsion is followed by gelling with an amount of polyvinyl alcohol suffiecient to produce a gel of the required properties.

The present invention is also directed to a method for treating wounds by the application of the disclosed and claimed compositions. In a preferred embodiment, the wound is treated with antioxidants for a period of from 3 to 7 days, with no other treament modality. Without meaning to be bound by mechanism, it is proposed that the treatment adjusts the level of reactive oxygen species in the wound to a level where further treatments can be effective. Then the antioxidant treatment is stopped, and treatment with other modalities, for example growth factors, is started.

The invention has been revealed in detail with particular reference to preferred embodiments thereof, but it will be understood by one skilled in the art that variations and modifications can be effected within the spirit and scope of the invention.

1. A composition for applying to a wound for promoting healing comprising:
(i) an antioxidant in which the active center is a 2,2,6,6-tetraalkyl piperidinyl group and in which the nitrogen atom in the piperidine ring in the active center is attached to a group selected from the group consisting of H (hydrogen), alkyl, O (oxygen radical), alkoxy, acyloxy, and hydroxyalkoxy, and
(ii) an inert carrier that comprises a hydrogel.

2. The composition of claim 1 in which the antioxidant is selected from the group consisting of;
bis(2,2,6,6-tetramethyl-4-piperidyl)sebacate,
bis(2,2,6,6-tetramethyl-4-piperidyl)succinate,
bis(1,2,2,6,6-pentamethyl-4-piperidyl)sebacate,
bis(1-octyloxy-2,2,6,6-tetramethyl-4-piperidyl)sebacate,
bis(1,2,2,6,6-pentamethyl-4-piperidyl) n-butyl-3,5-di-tert-butyl-4-hydroxybenzylmalonate,
the condensate of 1-(2-hydroxyethyl)-2,2,6,6-tetramethyl-4-hydroxypiperidine and succinic acid,
the condensate of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate,
tetrakis(2,2,6,6-tetramethyl-4-piperidyl)-1,2,3,4-butane-tetracarboxylate, 4-benzoyl-2,2,6,6-tetramethylpiperidine,
4-stearyloxy-2,2,6,6-tetramethylpiperidine,
bis(1,2,2,6,6-pentamethylpiperidyl)-2-n-butyl-2-(2-hydroxy-3,5-di-tert-but yl-benzyl)malonate,
3-n-octyl-7,7,9,9-tetramethyl-1,3,8-triazaspiro[4.5]decan-2,4-dion, bis(1-octyloxy-2,2,6,6-tetramethylpiperidyl)sebacate,
bis(1-octyloxy-2,2,6,6-tetramethylpiperidyl)succinate,
the condensate of N,N′-bis-(2,2,6,6-tetramethyl-4-piperidyl)hexamethylenediamine and 4-morpholino-2,6-dichloro-1,3,5-triazine,
the condensate of 2-chloro-4,6-bis(4-n-butylamino-2,2,6,6-tetramethylpiperidyl)-1,3,5-triazine and 1,2-bis(3-aminopropylamino)ethane,
the condensate of 2-chloro-4,6-di-(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4. 5]decane-2,4-dione, 3-dodecyl-1-(2,2,6,6-tetramethyl-4-piperidyl)pyrrolidin-2,5-dione, 3-dodecyl-1-(1,2,2,6,6-pentamethyl-4-piperidyl)pyrrolidine-2,5-dione,
a mixture of 4-hexadecyloxy- and 4-stearyloxy-2,2,6,6-tetramethylpiperidine,
the condensation product of N, N′-bis(2,2,6,6-tetramethyl-4-piperidyl)hexamethylenediamine and 4-cyclohexylamino-2,6-dichloro-1,3,5-triazine,
the condensation product of 1,2-bis(3-aminopropylamino)ethane and 2,4,6-trichloro-1,3,5-triazine as well as 4-butylamino-2,2,6,6-tetramethylpiperidine,
N-(2,2,6,6-tetramethyl-4-piperidyl)-n-dodecylsuccinimid, N-(1,2,2,6,6-pentamethyl-4-piperidyl)-n-dodecylsuccinimid, 2-undecyl-7,7,9,9-tetramethyl-1-oxa-3,8-diaza-4-oxo-spiro[4,5]decane, and
the reaction product of 7,7,9,9-tetramethyl-2-cycloundecyl-1-oxa-3,8-diaza-4-oxospiro [4,5]decane and epichlorohydrin.

3. The composition of claim 1 in which the carrier is a mixture of water and a polymer selected from the group consisting of polyacrylamide, polymethacrylic acid, copolymers of diacetone acrylamide (DAA) and hydroxyethyl acrylate (HEA), polylactic acid, polyglycolic acid, polyacrylonitrile, and polyvinyl acohol.

4. A composition for applying to a wound for healing comprising;
(i) an antioxidant in which the active center is a hindered phenol moiety, and
(ii) an inert carrier that comprises a hydrogel.

5. The composition of claim 4 in which said antioxidant is selected from the group consisting of:
tetrakismethylene(3,5-di-t-butyl-4-hydroxyhydrocinnamate)methane,
octadecyl 3-(3′,5′-di-t-butyl-4′-hydroxyphenyl)propionate,
2,2′-thiodiethylene bis(3-(3,5-di-t-butyl-4-hydroxyphenyl)propionate),
2,2′-ethylidenebis(4,6-di-t-butylphenol),
1,3,5-tris(3,5-di-t-butyl-4-hydroxybenzyl)isocyanurate,
1,3,5-trimethyl-2,4,6-tris(3,5-di-tert butyl-4-hydroxybenzyl) benzene,
2,2′-methylenebis(6-t-butyl-4-methylphenol),
4,4′-butylidenebis(2-t-butyl-5-methylphenol,
2,2′-isobutylidenebis(4,6-dimethylphenol),
2,5-di-t-amylhydroquinone,
1,1,3-tris(2′-methyl-4′-hydroxy-5′-t-butylphenyl)butane,
1,3,5-tris(4-t-butyl-3-hydroxy-2,6 dimethylbenzyl)-1,3,5-triazine-2,4,6-(1H, 3H, 5H)-trione,
N,N′-hexamethylene-bis(3-(3,5-di-t-butyl-4-hydroxyphenyl)propionamide),
4,4′-thiobis(2-t-butyl-4-methylphenol),
2,2′-methylenebis(4-methyl-6-(−1-methylcyclohexyl)phenol), and
triethylene glycol bis(30(3-tert-butyl-4-hydroxy-5-methylphenyl)propionate),

6. The composition of claim 4 in which the carrier is a mixture of water and a polymer selected from the group consisting of polyacrylamide, polymethacrylic acid, copolymers of diacetone acrylamide (DAA) and hydroxyethyl acrylate (HEA), polylactic acid, polyglycolic acid, polyacrylonitrile, and polyvinyl acohol.

7. A method for treating a wound in a vertebrate comprising the step of allowing the wound to come into contact with the composition of claim 1.

8. The method of claim 7 for treating a wound in a vertebrate further comprising the steps of
(i) allowing the the wound to remain in contact with the composition of claim 1 for a period of between 3 and 7 days,
(ii) applying to the wound further treatment modalities.

9. A method for treating a wound in a vertebrate comprising the step of allowing the wound to come into contact with the composition of claim 4.

10. The method of claim 9 for treating a wound in a vertebrate further comprising the steps of
(i) allowing the the wound to remain in contact with the composition of claim 4 for a period of between 3 and 7 days,
(ii) applying to the wound further treatment modalities.
